(19) United States
(12) Patent Application Publication
(10) Pub. No.: US 2012/0015388 A1
(43) Pub. Date: Jan. 19, 2012

(54) Diluent for Preparing Analytical Sample

(75) Inventors: Takeshi Takagi, Kyoto (JP); Toshikatsu Sakai, Kyoto (JP); Chiaki Miyamoto, Kyoto (JP); Takefumi Asami, Kyoto (JP); Tatsuo Kamata, Kyoto (JP); Takuya Yotani, Ryugasaki-shi (JP); Takayuki Oka, Tokyo (JP)

(73) Assignees: SEKISUI MEDICAL CO., LTD., Tokyo (JP); ARKRAY, Inc., Kyoto (JP)

(21) Appl. No.: 13/166,575

(22) Filed: Jun. 22, 2011

(30) Foreign Application Priority Data
Jun. 23, 2010 (JP) 2010-142245

(51) Int. Cl. C12Q 1/54; C12M 1/40

(52) U.S. Cl. 435/14; 435/288.1

(57) ABSTRACT

The ionic strength of a diluent for preparing an analytical sample is set to be 0.06 to 0.16. The analytical sample prepared by using the diluent having the ionic strength within this range can be subjected to both for analyzing a first object in a test sample by electrode method and for analyzing a second object in the test sample by liquid chromatography method, and high-precision measurement can be attained. The analytical sample is especially useful for preparing a sample for measurement used both for measuring glucose concentration in a blood sample by enzyme electrode method and for measuring glycohemoglobin concentration in the blood sample by liquid chromatography method.

### TECHNICAL FIELD

The present invention relates to a diluent for preparing an analytical sample, which diluent can be used both for analyzing a first object in a test sample by electrode method and for analyzing a second object in the test sample by liquid chromatography method.

### BACKGROUND ART

The method for analyzing a substance in a test sample such as a biological sample includes various methods such as electrode method and liquid chromatography method, and those methods are widely utilized in research and clinical sites.

The method for analyzing a substance by electrode method has been, for example, applied to the measurement of glucose concentration in blood by enzyme electrode method, which measurement is generally carried out in a screening test and treatment of diabetes (Japanese Laid-open Patent Application (Kokai) No. 9-33533, Japanese Laid-open Patent Application (Kokai) No. 2005-148058). According to this method, glucose in a blood sample is reacted with glucose oxidase (GOD), glucose dehydrogenase (GDH) or the like, an electric current generated by the resulting oxidation-reduction reaction of enzyme reaction product (e.g. hydrogen peroxide) or mediater such as potassium ferricyanide is measured, an operation such as equivalent point method (end point method) or differentiation (rate method) is performed to obtain the glucose concentration in the blood sample.

The method for analyzing a substance by liquid chromatography method has been, for example, applied to the measurement of glycohemoglobin concentration in blood by liquid chromatography method, which measurement is generally carried out in a screening test and treatment of diabetes (Japanese Laid-open Patent Application (Kokai) No. 5-5730, Japanese Laid-open Patent Application (Kokai) No. 9-178719). According to this method, components such as HbAlc and HbF are separated on the analytical column utilizing the difference in electric charge, and the glycohemoglobin concentration in a blood sample is obtained based on the chromatogram showing the relationship between elution times and the amounts of elution of each fraction (for example, optical information such as absorbance).

Conventionally, the above-described analysis by using electrode method and the above-described analysis by using liquid chromatography method have been carried out separately. For example, a blood analyzer for measuring glucose concentration by enzyme electrode method and a blood analyzer for measuring glycohemoglobin concentration by liquid chromatography method existed as separate apparatuses.

The present inventors sought to attain more excellent usefulness, and developed a blood analyzer which can measure both glucose concentration and glycohemoglobin concentration by using a prepared sample for measurement commonly (WO2008/035748).

### SUMMARY OF INVENTION

In cases where a common analytical sample is used in both an analysis by electrode method and an analysis by liquid chromatography method for a test sample, there may be a problem of analytical precision since the same diluent for preparing the sample is used.

For example, in a blood analyzer in which the aforementioned measurement of glucose concentration by enzyme electrode method and the aforementioned measurement of glycohemoglobin concentration by liquid chromatography method are carried out by using the common sample for measurement, the following problem arises. Firstly, GOD electrode method as used herein utilizes fact that an electric current passes between cathode and anode by decomposing glucose at a GOD-immobilized membrane, bringing hydrogen peroxide as an enzyme reaction product to a hydrogen peroxide electrode, and applying external voltage to cause an oxidation-reduction reaction. At this time, a diluent is a supporting electrolyte solution, so that the higher the ionic strength of the diluent, the better the detection performance.

On the other hand, in liquid chromatography method as used herein, components such as HbAlc and HbF are separated by utilizing the difference in electric charge depending on the types of glycohemoglobin, so that the higher the ionic strength of the diluent, the lower the separation performance.

Therefore, it was difficult to simultaneously attain the same diluent for preparing an analytical sample which is subjected to both analysis systems of an analysis of a first object in a test sample by electrode method and an analysis of a second object in the test sample by liquid chromatography method, and high analytical precision for both two objects.

In view of these circumstances, an object of the present invention is to provide a diluent which can prepare an analytical sample used both for analyzing a first object in a test sample by electrode method and for analyzing a second object in the test sample by liquid chromatography method, and can attain high-precision measurement.

To solve the above-described problem, the present inventors intensively studied to discover that a diluent having the ionic strength range of 0.06 to 0.16 is suited for the common diluent in both analysis systems of an analysis of a first object in a test sample by electrode method and an analysis of a second object in the test sample by liquid chromatography method, thereby completing the present invention.

That is, the present invention is as follows.

(1) A diluent for preparing an analytical sample, which diluent can be used both for analyzing a first object in a test sample by electrode method and for analyzing a second object in the test sample by liquid chromatography method, the diluent having an ionic strength of 0.06 to 0.16.

(2) The diluent according to item 1, wherein the first object in the test sample is analyzed by electrode method using an enzyme which reacts with the first object in the test sample.

(3) The diluent according to item 2, wherein the first object is glucose and the second object is glycohemoglobin, and

the glucose concentration in the test sample is measured by enzyme electrode method, and

the glycohemoglobin concentration in the test sample is measured by liquid chromatography method.

(4) The diluent according to any one of items 1 to 3, wherein the test sample is blood.

(5) The diluent according to any one of items 1 to 4, wherein an ion contained in the diluent is one or more selected from the group consisting of sodium ion, potassium ion, calcium ion, magnesium ion, chloride ion, phosphate ion and hydrogen carbonate ion.

(6) The diluent according to item 4 or 5, further comprising a hemolytic agent.

(7) The diluent according to item 6, wherein said hemolytic agent is one or more selected from the group consisting of saponin, polyoxyethylene alkyl aryl ether, higher aliphatic alcohol, alkyl aryl polyether alcohol, polyoxyethylene glycol or polyoxyethylene ether of sulfonate compound or sulfate compound, polyoxyethylene adduct of sorbitol fatty acid ester, and ammonium chloride.

(8) A kit for preparing an analytical sample, which kit can be used both for analyzing a first object in a blood sample by electrode method and for measuring a second object in the blood sample by liquid chromatography method, the kit comprising a diluent and a hemolytic agent, wherein an ionic strength of the diluent after mixed with the hemolytic agent is 0.06 to 0.16.

(9) A kit according to item 8, wherein said hemolytic agent is one or more selected from the group consisting of saponin, polyoxyethylene alkyl aryl ether, higher aliphatic alcohol, alkyl aryl polyether alcohol, polyoxyethylene glycol or polyoxyethylene ether of sulfonate compound or sulfate compound, polyoxyethylene adduct of sorbitol fatty acid ester, and ammonium chloride.

(10) An analytical method comprising the steps of:

mixing a diluent and a test sample to prepare the analytical sample;

a first analysis step of analyzing a first object in said analytical sample by electrode method; and

a second analysis step of analyzing a second object in said analytical sample by liquid chromatography method;

wherein the diluent being the diluent according to any one of items 1 to 7.

(11) The analytical method according to item 10, wherein

said first analysis step is a step of measuring glucose concentration in said analytical sample by enzyme electrode method, and

said second analysis step is a step of measuring glycohemoglobin concentration in said analytical sample by liquid chromatography method.

(12) An analytical apparatus having:

a diluent bottle containing a diluent for preparing an analytical sample;

a preparation device of the analytical sample, in which said diluent supplied from said diluent bottle and a test sample are mixed to prepare the analytical sample;

an analytical device of a first object, in which the first object is analyzed by electrode method for the analytical sample prepared in said preparation device of the analytical sample; and

an analytical device of a second object, in which the second object is analyzed by liquid chromatography method for the analytical sample prepared in said preparation device of the analytical sample,

said diluent being the diluent according to any one of items 1 to 7.

(13) The analytical apparatus according to item 12, wherein

said analytical device of the first object is for measuring glucose concentration in said analytical sample by enzyme electrode method, and

said analytical device of the second object is for measuring glycohemoglobin concentration in said analytical sample by liquid chromatography method.

(14) A method for preparation of an analytical sample both for analyzing a first object in a test sample by electrode method and for analyzing a second object in the test sample by liquid chromatography method comprising the step of mixing a diluent and the test sample,

wherein the diluent is the diluent according to claim 1.

According to the present invention, a diluent which can prepare an analytical sample used both for analyzing a first object in a test sample by electrode method and for analyzing a second object in the test sample by liquid chromatography method, and can attain high-precision measurement is provided.

### BRIEF DESCRIPTION OF THE DRAWINGS

FIG. 1 is a flow chart showing the analytical method according to the present invention.

FIG. 2 is a graph showing the influence by the ionic strength of the diluent on the measurement performance of glucose concentration and hemoglobin Alc.

### MODE FOR CARRYING OUT THE INVENTION

The diluent according to the present invention is used for preparing an analytical sample which can be used both for analyzing a first object in a test sample by electrode method and for analyzing a second object in the test sample by liquid chromatography method.

The above-described electrode method may be any method which can analyze a substance by utilizing an electrode. For example, enzyme electrode method by using an enzyme is exemplified, but the electrode method is not restricted thereto. In the above-described enzyme electrode method, as an enzyme immobilized on an electrode, the following enzymes are exemplified. In cases where the first object is glucose, examples of such enzymes include those generally used in the enzyme electrode method, such as glucose oxidase (GOD) and glucose dehydrogenase (GDH), and preferably GOD, but are not restricted thereto. In cases where the first object is lactic acid, examples of such enzymes include LDH (lactic acid dehydrogenase, lactate dehydrogenase), cytochrome c dependent D-lactate dehydrogenase, cytochrome c dependent L-lactate dehydrogenase, NAD(P)-dependent enzyme D-lactate dehydrogenase, NAD(P)-dependent enzyme L-lactate dehydrogenase, LCD (lactic oxidase, lactate oxidase), but are not restricted thereto.

The above-described liquid chromatography method can be preferably applied in cases where the second object is glycohemoglobin, but is not restricted thereto. The measurement of glycohemoglobin by liquid chromatography method is carried out by measuring an absorbance of each fraction at the absorption wavelength of hemoglobin after separating the fraction on the analytical column. Examples of the analytical column used include those generally used in separating glycohemoglobin, which filler is silica gel, polymethacrylate, polyhydroxymethacrylate, polyvinyl alcohol, styrene-divinylbenzene copolymer or the like.

In the present invention, the test sample contains the first object and the second object, and the examples of the test sample preferably include a blood sample, but are not restricted thereto.

In the dilution according to the present invention, the ionic strength is 0.06 to 0.16, preferably 0.08 to 0.14, more preferably 0.10 to 0.12. In the present invention, the ionic strength is one obtained by adding the products of mol concentration m of each ion and the square of charge z for all ionic species in solution, and then dividing the total into halves, and can be calculated by using the following equation (A).

I=(ΣCi·Zi²)/2(A)

Wherein, Ci and Zi represent the concentration and the charge of the “i”thion in the solution, respectively. In cases where the ionic strength is larger than 0.16, the second object cannot be well separated by the liquid chromatography column, and the analytical precision for the second object is poor. In cases where the ionic strength is lower than 0.06, the detection performance for the first object by the electrode method is poor. The test sample is diluted with the use of the diluent of the present invention until the usual dilution factor, for example, about 50 to 200 times is attained. The analytical sample prepared by using the diluent having the ionic strength within this range can be subjected to both for analyzing a first object in a test sample by electrode method and for analyzing a second object in the test sample by liquid, chromatography method, and high-precision analysis can be attained. The analytical sample is especially useful for preparing a sample for measurement used both for analyzing glucose concentration in a blood sample by enzyme electrode method and for analyzing glycohemoglobin concentration in the blood sample by liquid chromatography method.

The type of an ion contained in the diluent according to the present invention is not restricted, for example, the ion is one or more selected from the group consisting of sodium ion (Na⁺), potassium ion (K⁺), calcium ion (Ca²⁻), magnesium ion (Me²⁺), chloride ion (Cl⁻), phosphate ion (PO₄³⁻) and hydrogen carbonate ion (HCO₃⁻). This is because more stable measurement system can be obtained by using the same type of ion as that of ion contained in a living body in measuring a component in the blood specimen. The valency of the ion used herein is not restricted.

The diluent according to the present invention is prepared by using an electrolyte which generates the above-described ion, such as an alkali metal salt or alkaline-earth metal salt.

The diluent according to the present invention may contain a hemolytic agent in an amount of not adversely affecting the effect of the present invention. Examples of the hemolytic agent for destroying a blood cell membrane of a blood cell component in a blood specimen include known hemolytic agents including glycoside such as saponin; surfactant such as polyoxyethylene alkyl aryl ether, higher aliphatic alcohol, alkyl aryl polyether alcohol, polyoxyethylene glycol or polyoxyethylene ether of sulfonate compound or sulfate compound, and polyoxyethylene adduct of sorbitol fatty acid ester; and ammonium chloride. The hemolytic agent may be preliminarily added to the diluent according to the present invention, and it may be added together with the blood specimen in preparing a sample for measurement, or may be added before or after adding the blood specimen.

Further, the diluent according to the present invention may contain any additive such as a known antiseptic in an appropriate amount of not adversely affecting the effect of the present invention.

In the present invention, the diluent can constitute a kit for preparing an analytical sample in combination with a hemolytic agent or a solution comprising a hemolytic agent, which kit can be used both for analyzing a first object in a blood sample by electrode method and for measuring a second object in the blood sample by liquid chromatography method. In this case, a container containing the diluent and a container containing the hemolytic agent or the solution comprising the hemolytic agent are included as separate packagings in the kit. The ionic strength when the diluent and the hemolytic agent or the solution comprising the hemolytic agent are mixed is 0.06 to 0.16, preferably 0.08 to 0.14, more preferably 0.10 to 0.12.

By preparing an analytical sample with the use of this kit, the analytical sample can be subjected to both for analyzing a first object in a blood sample by electrode method and for analyzing a second object in the blood sample by liquid chromatography method.

The diluent according to the present invention can be applied to a method for preparation of an analytical sample both for analyzing a first object in a test sample by electrode method and for analyzing a second object in the test sample by liquid chromatography method comprising the step of mixing a diluent and the test sample.

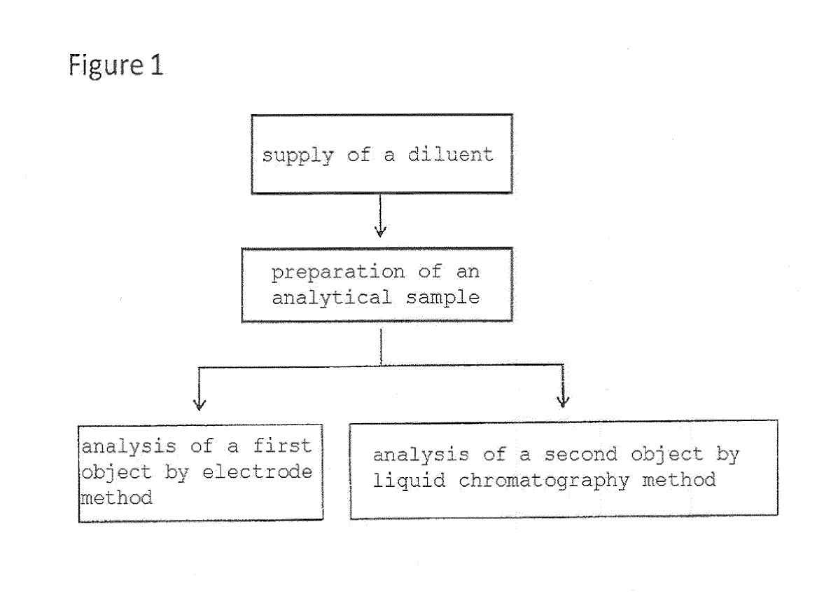

The diluent according to the present invention also can be applied to an analytical method comprising the steps of: (i) mixing a diluent for preparing an analytical sample and a test sample to prepare the analytical sample; (ii) a first analysis step of analyzing a first object in the analytical sample by electrode method; and (iii) a second analysis step of analyzing a second object in the analytical sample by liquid chromatography method. As for the analytical method, see the flow chart shown in FIG. 1. In the analytical method, the preferable case is that the first analysis step (ii) is a step of measuring glucose concentration in the analytical sample by enzyme electrode method, and the second analysis step (iii) is a step of measuring glycohemoglobin concentration in the analytical sample by liquid chromatography method, but the present invention is not restricted thereto. High-precision analysis can be attained for both the first object and the second object in the test sample by carrying out the above-mentioned analytical method with the use of the dilution of the present invention as the diluent in the step (i) of preparing the analytical sample.

The diluent according to the present invention can be applied to analyze the first object and the second object by using an analytical apparatus having a diluent bottle, a preparation device of the analytical sample, an analytical device of the first object and an analytical device of the second object.

The diluent bottle used herein contains the diluent of the present invention. In the preparation device of the sample, the diluent of the present invention supplied from the diluent bottle and a test sample are mixed to prepare the analytical sample. In the analytical device of the first object, the first object is analyzed by electrode method for the analytical sample prepared in the preparation device of the sample. In the analytical device of the second object, the second object is analyzed by liquid chromatography method for the analytical sample also prepared in the preparation device of the sample. In this analytical apparatus, the diluent according to the present invention functions as the diluent for preparing an analytical sample, which diluent is used both for analyzing the first object in a test sample by electrode method and for analyzing the second object in the test sample by liquid chromatography method. The analytical apparatus used herein preferably includes the apparatus wherein the analytical device of the first object is for measuring glucose concentration in the analytical sample by enzyme electrode method, and the analytical device of the second object is for measuring glycohemoglobin concentration in the analytical sample by liquid chromatography method, but is not restricted thereto.

The diluent according to the present invention also serves as a washing solution for a path and a measuring device in this analytical apparatus.

### EXAMPLES

The present invention will now be described in more detail by way of Examples, but the present invention is not restricted to these Examples.

### Example 1

The influence of the ionic strength of the diluent for preparing the test sample on the analytical performance of the first object and the second object was investigated. In this Example, the first object was glucose, the second object was glycohemoglobin, and each concentration of these in the test blood sample was measured by the following method to evaluate the measurement performance.

Buffers containing supporting electrolytes (alkali metal, alkaline-earth metal and the like) having different concentrations were prepared, and diluents having various ionic strengths were prepared. The glucose concentrations in the samples for measurement obtained by diluting the test blood sample with the use of these each diluent were measured by using GOD electrode. Further, the glycohemoglobin Alc concentrations in the samples for measurement were measured by liquid chromatography method using ion-exchange column. As for measuring apparatuses, the apparatuses widely used in the usual measurement were used.

As the test blood sample used herein, the sample in which both glucose concentration and glycohemoglobin Alc concentration were known was used, and the glucose concentration and the glycohemoglobin Alc concentration were 100 mg/dL and 5%, respectively. The difference between the measured value by the above-described measurement and the known concentration value was calculated as measurement accuracy (%). In cases where the ranges of the measurement accuracy are 97 to 103% for glucose concentration, and 99 to 101% for glycohemoglobin Alc concentration, it is considered that the accuracy of measurement is high. The reason why different criteria are set for each item is that glucose is greatly influenced by clinical condition of a sample donor of short time period on the order of hours to days, while glycohemoglobin Alc indicates clinical condition of a sample donor of medium- to long-term period on the order of several months and does not vary in the short time period.

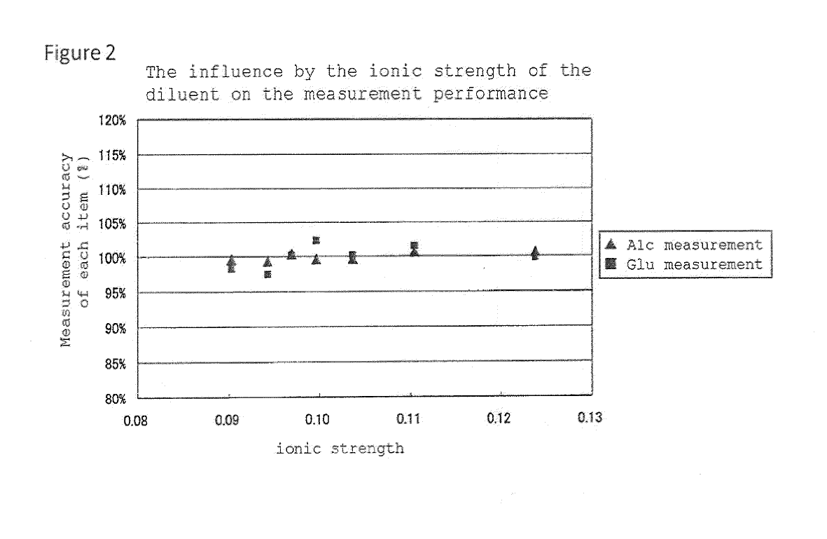

The result of each of the measurement accuracy for glucose and glycohemoglobin at each value of ionic strength of the diluent is shown in FIG. 2. By this, it can be seen that, the measurement accuracies for both glucose concentration and glycohemoglobin Alc concentration are high over the ionic strength range of the dilution from 0.090 to 0.124, and moreover, the measurement accuracies are especially high over the ionic Strength range from 0.100 to 0.120.

While the invention has been described in detail with reference to preferred embodiments thereof, it will be apparent to one skilled in the art that various changes can be made, and equivalents employed, without departing from the scope of the invention. All the cited references herein are incorporated as a part of this application by reference.

### INDUSTRIAL AVAILABILITY

By using the diluent according to the present invention, the diluent can be subjected to both for analyzing the first object in a test sample by electrode method and for analyzing the second object in the test sample by liquid chromatography method, and high-precision analysis can be attained, which is industrially useful. Especially, in cases where the diluent according to the present invention is applied for preparing a sample for measurement used both for measuring glucose concentration in a blood sample by enzyme electrode method and for measuring glycohemoglobin concentration in the blood sample by liquid chromatography method, the diluent is useful for a screening test and treatment of diabetes.

1. A diluent for preparing an analytical sample, which diluent can be used both for analyzing a first object in a test sample by electrode method and for analyzing a second object in the test sample by liquid chromatography method, wherein the diluent has an ionic strength of 0.06 to 0.16.

2. The diluent according to claim 1, wherein the first object in the test sample is analyzed by electrode method using an enzyme which reacts with the first object in the test sample.

3. The diluent according to claim 2, wherein the first object is glucose and the second object is glycohemoglobin, and
the glucose concentration in the test sample is measured by enzyme electrode method, and
the glycohemoglobin concentration in the test sample is measured by liquid chromatography method.

4. The diluent according to claim 1, wherein the test sample is blood.

5. The diluent according to claim 1, wherein said diluent contains one or more ions selected from the group consisting of sodium ion, potassium ion, calcium ion, magnesium ion, chloride ion, phosphate ion and hydrogen carbonate ion.

6. The diluent according to claim 4, further comprising a hemolytic agent.

7. The diluent according to claim 6, wherein said hemolytic agent is one or more selected from the group consisting of saponin, polyoxyethylene alkyl aryl ether, higher aliphatic alcohol, alkyl aryl polyether alcohol, polyoxyethylene glycol or polyoxyethylene ether of sulfonate compound or sulfate compound, polyoxyethylene adduct of sorbitol fatty acid ester, and ammonium chloride.

8. A kit for preparing an analytical sample, which kit can be used both for analyzing a first object in a blood sample by electrode method and for measuring a second object in the blood sample by liquid chromatography method, the kit comprising a diluent and a hemolytic agent, wherein an ionic strength of the diluent after mixed with the hemolytic agent is 0.06 to 0.16.

9. The kit according to claim 8, wherein said hemolytic agent is one or more selected from the group consisting of saponin, polyoxyethylene alkyl aryl ether, higher aliphatic alcohol, alkyl aryl polyether alcohol, polyoxyethylene glycol or polyoxyethylene ether of sulfonate compound or sulfate compound, polyoxyethylene adduct of sorbitol fatty acid ester, and ammonium chloride.

10. An analytical method comprising the steps of:
mixing a diluent and a test sample to prepare the analytical sample;
a first analysis step of analyzing a first object in said analytical sample by electrode method; and
a second analysis step of analyzing a second object in said analytical sample by liquid chromatography method;
wherein the diluent is the diluent according to claim 1.

11. The analytical method according to claim 10, wherein
said first analysis step is a step of measuring glucose concentration in said analytical sample by enzyme electrode method, and
said second analysis step is a step of measuring glycohemoglobin concentration in said analytical sample by liquid chromatography method.

12. An analytical apparatus having:
a diluent bottle containing a diluent for preparing an analytical sample;
a preparation device of the analytical sample, in which said diluent supplied from said diluent bottle and a test sample are mixed to prepare the analytical sample;
an analytical device of a first object, in which the first object is analyzed by electrode method for the analytical sample prepared in said preparation device of the analytical sample; and
an analytical device of a second object, in which the second object is analyzed by liquid chromatography method for the analytical sample prepared in said preparation device of the analytical sample,
wherein the diluent is the diluent according to claim 1.

13. The analytical apparatus according to claim 12, wherein
said analytical device of the first object is for measuring glucose concentration in said analytical sample by enzyme electrode method, and
said analytical device of the second object is for measuring glycohemoglobin concentration in said analytical sample by liquid chromatography method.
